(19) United States
(12) Patent Application Publication
(10) Pub. No.: US 2026/0232968 A1
(43) Pub. Date: Aug. 13, 2026

(54) INTRAVASCULAR DEVICE WITH INTERSPERSED BEAM PATTERN

(71) Applicant: SCIENTIA VASCULAR, INC., West Valley City, UT (US)

(72) Inventors: Brandon Rodney POPE, Salt Lake City, UT (US); Kenneth SMITH, West Valley City, UT (US); William Rigby ECCLES, Centerville, UT (US)

(21) Appl. No.: 19/534,559

(22) Filed: Feb. 9, 2026

Related U.S. Application Data
(60) Provisional application No. 63/756,691, filed on Feb. 10, 2025.

(51) Int. Cl. A61M 25/09

(52) U.S. Cl. CPC A61M 25/09; A61M 2025/09141; A61M 2025/0915

(57) ABSTRACT

The present disclosure relates to intravascular devices comprising a tube structure that includes cut patterns that can advantageously provide smooth transitions between different sections of the tube. The tube includes a plurality of cuts to increase the flexibility of the tube. The tube includes a proximal section with cuts of a first type, a distal section with cuts of a second type, and a transition section where cuts of both the first type and second type are interspersed with one another to provide a smooth transition with respect to bending flexibility, torquability, and/or columnar stiffness from the proximal section to the distal section.

### CROSS-REFERENCE TO RELATED APPLICATIONS

This application claims priority to and the benefit of U.S. Provisional Patent Application No. 63/756,691, filed Feb. 10, 2025 and titled “Intravascular Device with Interspersed Beam Pattern”, the entirety of which is incorporated herein by reference.

### BACKGROUND

Guidewire devices are often used to lead or guide catheters or other interventional devices to a targeted anatomical location within a patient's body. Typically, guidewires are passed into and through a patient's vasculature in order to reach the target location, which may be at or near the patient's heart or neurovascular tissue, for example. Radiographic imaging is often utilized to assist in navigating a guidewire to the targeted location. In many instances, a guidewire is left in place within the body during the interventional procedure where it can be used to guide multiple catheters or other interventional devices to the targeted anatomical location.

The flexibility of a guidewire device, particularly at the distal sections of the device, can affect performance. In many circumstances, relatively high levels of flexibility are desirable to provide sufficient lateral bending of the guidewire to enable effective passage through the tortuous bends and curves of a vasculature passageway while passing to the targeted area. For example, directing a guidewire to portions of the neurovasculature requires passage of the guidewire through curved passages such as the carotid siphon and other tortuous regions.

Another concern related to guidewire devices is the ability to effectively transmit torque from the proximal portion to the distal portion (i.e., the “torquability” of the guidewire device). As a greater length of a guidewire is passed into and through the vasculature, the amount of frictional surface contact between the guidewire and the vasculature increases, hindering easy movement of the guidewire through the vasculature passage. A guidewire with good torquability effectively transmits torque forces applied at the proximal end to the distal end of the device.

Some guidewire devices include a tube positioned over a distal section of the guidewire core. Because torsional forces are primarily transmitted through the outer sections of a cross-section of a member, the tube provides a path for effective transmission of torque, without the stiffness of a solid member of similar outer diameter. Typically, such tubes are formed from a superelastic material such as nitinol.

Some tubes include cuts intended to increase flexibility while preserving sufficient torquability. While such guidewire devices have many benefits, limitations remain. For example, conventional cut patterns can impart abrupt transitions in bending flexibility, torquability, and/or columnar stiffness to the guidewire device.

### SUMMARY

The present disclosure relates to guidewire devices with micro-fabricated tubing having various cut patterns as described herein. In one embodiment, a guidewire device includes a core having a proximal section and a distal section. A tube is coupled to the core such that the distal section extends into the tube. Cuts extend transversely into the tube to form a series of “beams” substantially extending in an axial direction and circumferentially extending “rings”. Each beam connects two adjacent rings.

The tube includes a proximal section, a distal section, and a transition section between the proximal and distal sections. The proximal section of the tube includes a cut pattern where the cuts form two or more beams between adjacent rings (i.e., two-beam cuts). The distal section of the tube includes a cut pattern where the cuts form a single beam between adjacent rings (i.e., one-beam cuts). The transition section includes a first type of cut and a second type of cut interspersed with one another. The first type of cut forms a single beam between adjacent rings (i.e., one-beam cuts) and the second type of cut forms two beams between adjacent rings (i.e., two-beam cuts).

In some embodiments, in the transition section, the first type of cut (i.e., one-beam cuts) and second type of cut (i.e., two-beam cuts) are interspersed in successive units wherein each successive unit comprises 1-3 successive cuts of the first type (i.e., one-beam cuts) and then 1-3 successive cuts of the second type (i.e., two-beam cuts), or vice versa.

In some embodiments, the units closer to the proximal section include more cuts of the second type (i.e., two-beam cuts) than cuts of the first type (i.e., one-beam cuts). In some embodiments, the successive units farther from the proximal section include more cuts of the first type (i.e., one-beam cuts) than cuts of the second type (i.e., two-beam cuts). For example, the transition section of the tube can include a series of multiple units where those that are closest to the proximal section comprise more cuts of the second type (i.e., two-beam cuts) than cuts of the first type (i.e., one-beam cuts). Then, continuing in the distal direction toward the distal section of the tube, subsequent units can increase the proportion of cuts of the first type (i.e., one-beam cuts).

At the distal section of the guidewire device, high bending flexibility and low columnar stiffness are generally desirable for reasons of safety and steerability. However, it is beneficial for more proximal sections of the device to have higher torquability and columnar stiffness to promote navigability through the vasculature. Two-beam cut patterns generally exhibit lower bending flexibility but higher torquability and columnar stiffness, whereas one-beam cut patterns generally exhibit higher bending flexibility but lower torquability and columnar stiffness.

The interspersed arrangement of cut types in the transition section beneficially provides a smooth transition from the two-beam cuts of the proximal section to the one-beam cuts of the distal section. The transition section, as disclosed herein, can minimize abrupt changes in bending flexibility, torquability, and/or columnar stiffness when transitioning from a two-beam cut pattern to a one-beam cut pattern.

Additional features and advantages will be set forth in part in the description that follows, and in part will be obvious from the description, or may be learned by practice of the embodiments disclosed herein. The objects and advantages of the embodiments disclosed herein will be realized and attained by means of the elements and combinations particularly pointed out in the appended claims. It is to be understood that both the foregoing brief summary and the following detailed description are exemplary and explanatory only and are not restrictive of the embodiments disclosed herein or as claimed.

### BRIEF DESCRIPTION OF THE DRAWINGS

A more particular description of the invention briefly described above will be rendered by reference to specific embodiments which are illustrated in the appended drawings. Understanding that these drawings depict only typical embodiments of the invention and are not limiting of its scope, the invention will be described and explained with additional specificity and detail through the use of the accompanying drawings in which:

FIGS. 1A and 1B illustrate an example embodiment of a guidewire device;

FIGS. 2A-2E illustrate a one-beam cut pattern that can be included in the tube of the guidewire device of FIG. 1;

FIGS. 3A and 3B illustrate a two-beam cut that can be included in the tube of the guidewire device of FIG. 1;

FIGS. 3C and 3D illustrate a ring and beam configuration resulting from a spiral cut pattern;

FIGS. 4A and 4B illustrate an embodiment of a tube including a distal section comprising a one-beam cut pattern and a proximal section having a two-beam cut pattern, showing that an abrupt change in bending flexibility, torque transmissibility, and/or columnar stiffness can occur;

FIG. 5A illustrates an embodiment of a tube comprising a cut pattern that includes interspersed one-beam and two-beam cuts to function as an effective transition section between a two-beam section and a one-beam section;

FIGS. 5B-5G illustrate units in which one-beam and two-beam cuts may be interspersed in the tube of FIG. 5A;

FIG. 6 illustrates a section of tube showing how an interspersed configuration can affect the bending radius in a preferred bending direction.

### DETAILED DESCRIPTION

### Introduction

The present disclosure relates to guidewire devices providing effective transitions between sections of a tube with different cut patterns. The ability to safely steer and route a guidewire to a targeted anatomical location can be improved by providing a tube with varying levels of torquability, columnar stiffness, and bending flexibility. This can be achieved by providing a tube with different sections defined by different cut patterns, each of which can be tailored to desired balances of such mechanical parameters. However, the transition point where the cut pattern changes may cause an abrupt change in the mechanical characteristics of the tube. These abrupt changes in mechanical characteristics may negatively affect the performance of the guidewire device and are typically undesirable.

The severity of these transition points (i.e., how abrupt a transition point is) can be minimized using alterations in the microfabricated cut patterns as they approach the transition point. For example, the severity of a transition point from one cut pattern to the next can be minimized as described in U.S. Pat. No. 11,951,267, which is incorporated herein by reference in its entirety. As shown and described in relation to FIG. 10 of U.S. Pat. No. 11,951,267, the abruptness of a transition from a two-beam section to a one-beam section can be minimized by adjusting the axial thickness of rings so that rings of the two-beam section near the transition have smaller axial thickness and rings of the one-beam section near the transition have greater axial thickness. While this approach can beneficially avoid abrupt changes in bending flexibility/stiffness, it has not proven as effective in avoiding changes in torquability and/or columnar stiffness. In contrast, the use of a transition section including interspersed cut types, as disclosed herein, can beneficially minimize abrupt transition points with respect to bending flexibility, torquability, and/or column stiffness.

While many of the specific examples disclosed herein relate to guidewire devices, it will be understood that the same features can be applied to other intravascular devices, such as catheters, that make use of a tubular structure.

### Intravascular Device Overview

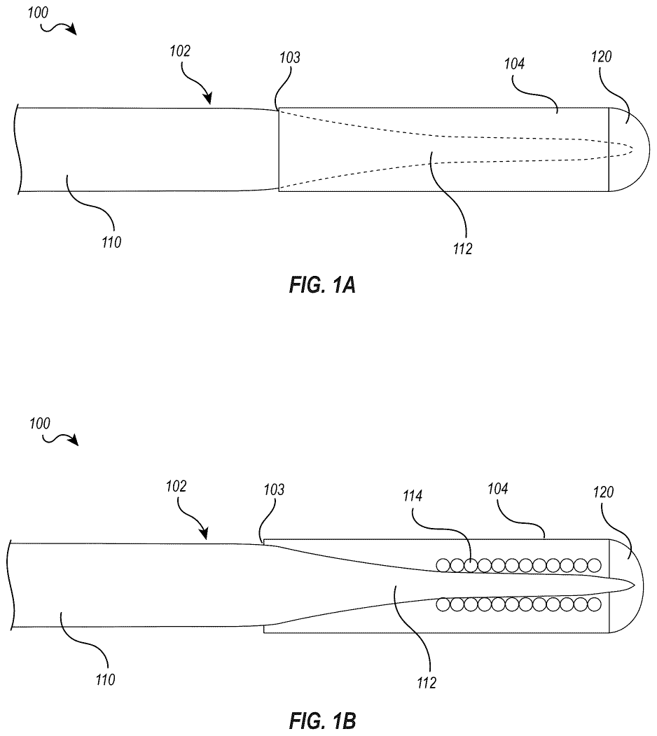

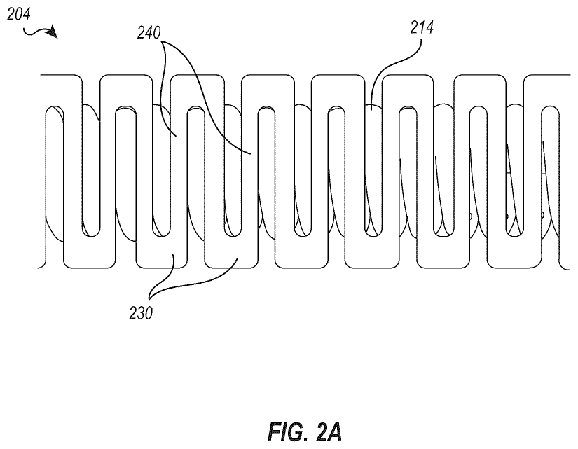

FIGS. 1A and 1B illustrate an exemplary guidewire device 100 having a core 102. A tube 104 is coupled to the core 102 and extends distally from a point of attachment 103 to the core 102. As shown, a distal section of the core 102 extends into the tube 104 and is surrounded by the tube 104. In some embodiments, the core 102 includes one or more tapering sections so that the core 102 can fit within and extend into the tube 104. For example, the distal section of the core 102 may be ground to progressively taper to a smaller diameter at the distal end. In this example, the core 102 and the tube 104 have substantially similar outer diameters at the attachment point 103 where they adjoin and attach to one another. In some embodiments, the core 102 and the tube 104 have different outer diameters at the attachment point 103 where they adjoin and attach to one another, with the difference in diameter being compensated for by a weld, solder, adhesive, collet, bushing, or other means of structural attachment.

The tube 104 can be coupled to the core 102 (e.g., using adhesive, soldering, and/or welding) in a manner that allows torsional forces to be transmitted from the core 102 to the tube 104 and thereby to be further transmitted distally by the tube 104. A medical grade adhesive 120 may be used to couple the tube 104 to the core 102 at the distal end of the device and to form an atraumatic covering. As explained in more detail below, the tube 104 includes a plurality of cuts. The cuts can be arranged to form a cut pattern which beneficially provides for effective shapeability near the distal tip of the guidewire device 100 while also maintaining good torquability and columnar stiffness. For clarity, cut patterns are not shown in FIG. 1 but are described in more detail below.

The proximal section 110 of the guidewire device 100 extends proximally to a length necessary to provide sufficient guidewire length for delivery to a targeted anatomical area. The proximal section 110 typically has a length ranging from about 50 to 350 cm. The proximal section 110 may have a diameter of about 0.014 inches, or a diameter within a range of about 0.008 to 0.125 inches. The distal section 112 of the core 102 may taper to a diameter of about 0.002 inches, or a diameter within a range of about 0.001 to 0.050 inches. In some embodiments, the tube 104 has a length within a range of about 3 to 100 cm.

In some embodiments, the distal section 112 of the core 102 tapers to a round cross-section. In other embodiments, the distal section 112 of the core 102 has a flat or rectangular cross-section. The distal section 112 may also have another cross-sectional shape, such as another polygon shape, an ovoid shape, an erratic shape, or combination of different cross-sectional shapes at different areas along its length.

One or more coils 114 can be positioned upon the distal section 112 of the core 102 to be positioned between the core 112 and the inner surface of the tube 104. The device 100 can incorporate the coil arrangements described in U.S. patent application Ser. No. 17/154,777, which is incorporated herein by reference in its entirety, including the arrangements of distal coil, proximal coil, and busing coil described therein.

### Cut Pattern Types

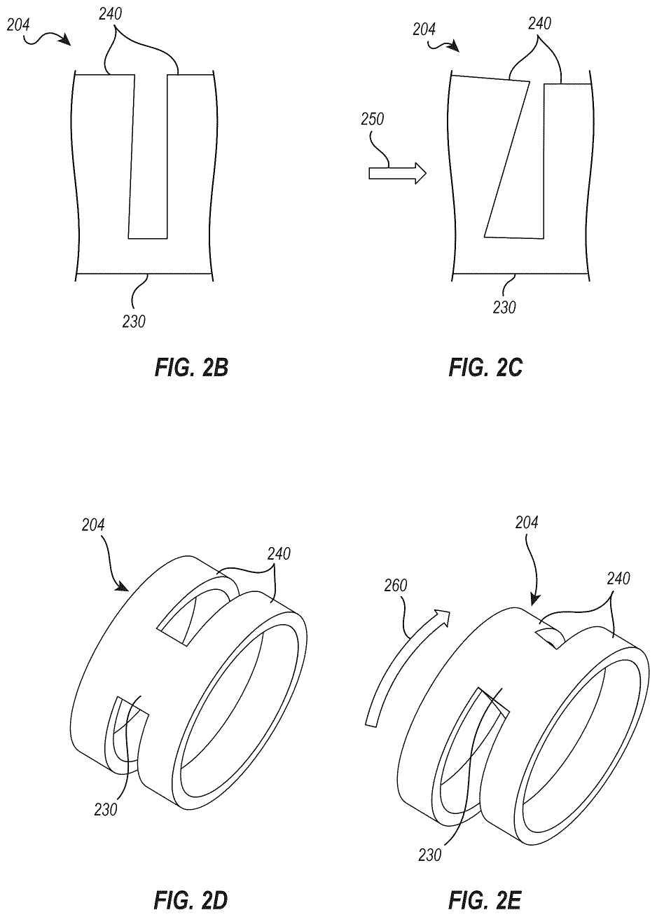

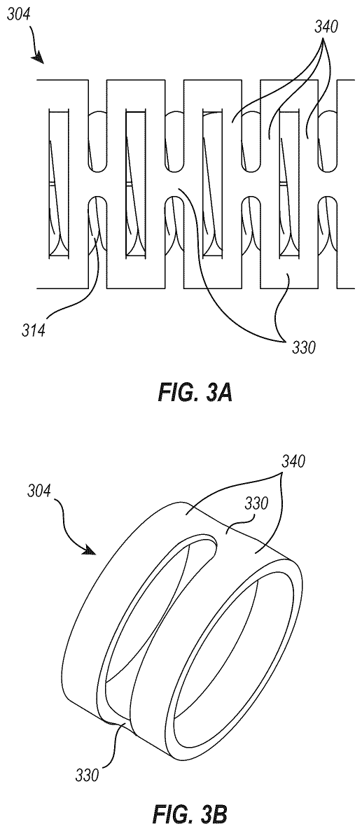

The tube 104 may include a plurality of cuts to provide increased bending flexibility compared to a tube without cuts. One such cut type is referred to herein as a “one-beam” cut. As illustrated in FIG. 2A, a one-beam cut is a cut that does not have an opposing cut directly opposite of it with respect to the longitudinal axis of the tube, thereby leaving a single beam 230 of longitudinally extending material between rings 240 of transversely and circumferentially extending material. The section of tube 204 shown in FIG. 2A also includes a coil 214 within the lumen of the tube 204.

In the illustrated section of tube 204, each successive beam 230 at each successive longitudinal cut location is rotated by 180 degrees from the previous beam 230. That is, the beams 230 exhibit a 180 degree “rotational offset”. Other rotational offsets may additionally or alternatively be utilized. For example, the cut pattern may include a rotational offset of 10, 20, 30, 40, 50, 60, 70, 80, 90, 100, 110, 120, 130, 140, 150, 160, 170, or 180 degrees, or a rotational offset within a range that uses any two of the foregoing values as endpoints.

In some embodiments, a standard rotational offset of 180 degrees is applied from one longitudinal cut position to the next to form a “segment” of two longitudinal cut positions, with each segment being rotationally offset from the previous segment by a different (not 180 degree) value according to any of the foregoing rotational offset values or ranges. In such embodiments, the two beams 230 within each segment are rotationally offset by 180 degrees, while at the segment-to-segment level, a different rotational offset may be applied.

In some embodiments, a section of one-beam cuts does not include a rotational offset, resulting in the beams 230 being aligned to form a “spine” along one circumferential side of the tube 204.

FIGS. 2B-2E show additional views of one-beam cuts at a single longitudinal cut location in perspective view.

FIGS. 2B and 2C illustrates the effect of a one-beam cut on bending flexibility. The space opened in the tube by the one-beam cut allows the tube to bend between the rings 240, increasing the bending flexibility. For similar reasons, a tube with one-beam cuts exhibits less columnar stiffness. For example, when force 250 is applied to the tube in an axial direction, the force 250 is transmitted from ring to ring through only one beam 240. As a result, the portion of the rings 240 that are not supported by the beam 230 may collapse towards one another such that at least some of the force 250 applied in the axial direction is not fully transmitted to more distal sections of the tube 204.

FIGS. 2D and 2E illustrates the effect of a one-beam cut on torquability. Because one-beam cuts result in the formation of only one beam 230 between the rings 240, torque 260 applied to a tube with one-beam cuts is transmitted from ring to ring through only one beam 240. As a result, the rings 240 can rotate laterally with respect to one another. Accordingly, at least some torque 260 applied to a tube in an angular direction is not fully transmitted to more distal sections of the tube 204.

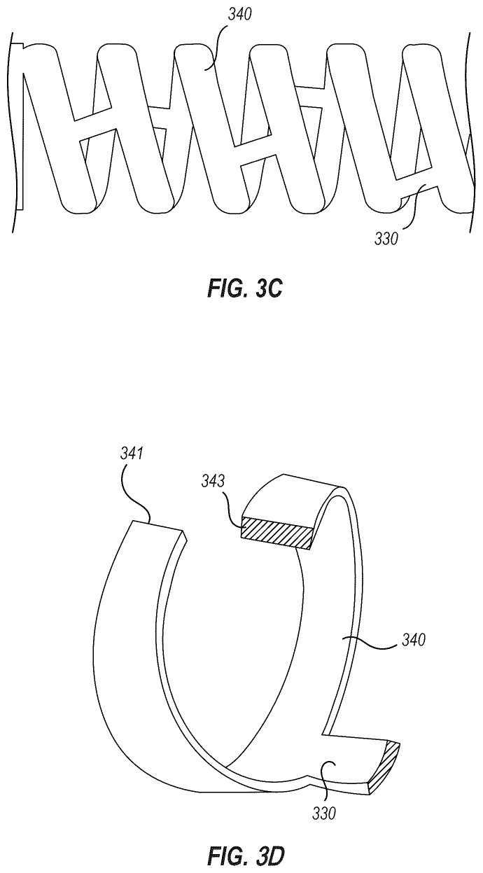

Another type of cut is referred to herein as a “two-beam cut.” As illustrated by the section of tube 304 shown in FIG. 3A, a two-beam cut comprises two opposing cuts with respect to the longitudinal axis of the tube, thereby leaving a pair of beams 330 of longitudinally extending material between rings 340 of transversely and circumferentially extending material. The section of tube 304 shown in FIG. 3A also includes a coil 314 within the lumen of the tube 304. Note that, in the view of FIG. 3A, at the locations where a single beam 330 is displayed along the centerline of the tube 304, it will be understood that corresponding beams 330 are circumferentially opposite the visible beams 330 and are hidden from view on the other side of the coil 314. For other longitudinal cut locations, the pair of corresponding beams 330 are clearly shown as circumferentially opposite one another along the “top” and “bottom” of the tube 304.

In the illustrated embodiment, the pair of beams 330 at each longitudinal cut position are symmetrically spaced from one another by 180 degrees. Other embodiments may additionally or alternatively include other circumferential spacing of beam pairs, such as 30, 60, 90, 120, or 150 degrees, or be within a range that uses any two of the foregoing values as endpoints.

As also shown in the illustrated embodiment, the successive beam pairs from one longitudinal cut location to the next are rotated by 90 degrees from the previous beam pair. Other rotational offsets may additionally or alternatively be utilized. For example, the cut pattern may include a rotational offset of 10, 20, 30, 40, 50, 60, 70, 80, or 90 degrees, or a rotational offset within a range that uses any two of the foregoing values as endpoints.

In some embodiments, a standard rotational offset of 90 degrees is applied from one longitudinal cut position to the next to form a “segment” of two longitudinal cut positions, with each segment being rotationally offset from the previous segment by a different (not 90 degree) value according to any of the foregoing rotational offset values or ranges. In such embodiments, the first beam pair and second beam pair within each segment are rotationally offset by 90 degrees, while at the segment-to-segment level, a different rotational offset may be applied.

FIG. 3B shows another view of the two-beam cut, showing the features of a single longitudinal cut location in perspective view.

Like one-beam cuts, two-beam cuts open spaces within the tube (i.e., the space between rings 340) allowing the tube to bend more easily. As compared to one-beam cuts, two-beam cuts maintain more of the structure of the tube and therefore maintain more of the original bending stiffness, columnar stiffness, and torquability of the tube, all else being equal. Accordingly, two-beam cuts may provide a section of a tube with increased bending flexibility when compared to a tube without cuts, but less bending flexibility than a tube with one-beam cuts. Accordingly, all else being equal (tube materials, cut width, cut spacing, cut depth, etc.), a two-beam cut pattern provides more torquability and columnar stiffness in a section of tube compared to an otherwise similar section tube with one-beam cuts.

The same concept extends to cut patterns with more than two beams between adjacent rings, such as three-beam cut patterns and four-beam cut patterns. Accordingly, all else being equal (tube materials, cut width, cut spacing, cut depth, etc.), a section of tube with a three-beam cut pattern has less bending flexibility, but greater columnar stiffness and torquability, than a two-beam cut pattern.

Although many of the disclosed examples include two-beam cuts and one-beam cuts, it will be understood that the same concepts may be applied to embodiments that incorporate one or more different cut patterns. That is, while the example transition sections disclosed herein intersperse two-beam cuts and one-beam cuts to provide a transition from a two-beam section to a one-beam section, the same interspersing of cut types can be used to transition between other cut types. For example, a transition section can provide a transition from a three-beam section to a two-beam section by interspersing three-beam cuts and two-beam cuts according to the interspersing concepts disclosed herein.

Cut patterns can further be defined based on the circumferential/rotational offset (or lack thereof) applied from one longitudinal cut location to the next, or from one segment of multiple longitudinal cut locations to the next. U.S. Pat. No. 11,369,351, which is incorporated herein by reference in its entirety, describes linear, helical, distributed, imperfect ramp, and sawtooth patterns. The cut patterns disclosed herein can include one or more of such circumferential/rotational patterns. The circumferential/rotational patterns can be selected and implemented to impose or avoid the formation of preferred bending planes within the tube, as desired.

In the examples of FIGS. 2A through 3B, the cuts are made at a perpendicular angle relative to the longitudinal axis of the tube. This results in beams that extend in a direction parallel to the longitudinal axis, and rings that include planar sides (i.e., the sides facing proximally and distally) that are perpendicular to the longitudinal axis. The illustrated rings 240, 340 in FIGS. 2A through 3B are also “enclosed” rings. That is, for a given enclosed ring, the ring can be said to “start” at an arbitrary circumferential location, extend around the circumference of the tube, and “end” where it started at the same axial location of the tube.

Other embodiments can include a cut pattern where: beams extend in a direction that is not parallel to the longitudinal axis of the tube, rings include planar sides that are not perpendicular to the longitudinal axis of the tube, and/or rings are not fully enclosed. For example, a cut pattern that extends around the tube in the shape of a spiral, with spaced interruptions to form the beams, can result in a configuration of rings and beams where the beams extend at an angle slightly offset from the longitudinal axis, with the angle depending on the pitch of the spiral. The rings of such a cut pattern would begin and end at different axial locations along the tube due to the pitch of the spiral cut. That is, such a ring can be said to start at an arbitrary circumferential location, extend around the circumference (e.g., 360 degrees), and end at a location that is slightly axially offset from the start position due to the pitch of the spiral cut. The planar sides of such a ring can also be angled at a non-perpendicular angle relative to the longitudinal axis of the tube and can instead be based on the pitch of the spiral cut.

FIG. 3C illustrates an example of such a cut pattern. In the embodiment of FIG. 3C, a spiral shaped cut is formed in the tube with interruptions that leave beams 330 connecting adjacent rings 340. FIG. 3D illustrates a partial sectional view of an example of a single ring 340 resulting from such a cut pattern. In the illustrated example, the ring 340 begins at an arbitrary start circumferential location 341, extends around the circumference (e.g., 360 degrees), and ends at an end circumferential location 343 that is slightly axially offset from the start circumferential location 341 due to the pitch of the spiral cut. As shown, the resulting ring 340 is not enclosed. The planar sides of the ring 340 also follow the spiral shape of the cut are therefore not perpendicular to the longitudinal axis of the tube. A beam 330 is also shown extending from the ring 340. The cut pattern shown in FIG. 3C is formed as a series of rings 340 and beams 330 shown in FIG. 3D.

A pattern made up of rings such as ring 340 shown in FIG. 3D would form a one-beam cut pattern. That is, one beam 330 extends between every 360 degrees of circumferential extension of each ring 340. A two-beam cut pattern would include two beams 330 extending between every 360 degrees of circumferential extension of each ring 340. In embodiments where the spacing of the beams 330 does not follow a repeating pattern matched to 360 degrees of rotation, the beam pattern can be referred to using fractional values. For example, beams 330 that are spaced every 180 degrees would coincide with a two-beam cut pattern, whereas beams that are spaced every 240 degrees can be referred to as a 1.5 beam cut pattern. Alternatively, it can be referred to as a pattern that includes one-beam portions and two-beam portions depending on the particular 360 degree section examined.

### Cut Pattern Transitions

Tubes formed using one or more sections that include one-beam cuts as shown in FIGS. 2A-2C can be beneficial. At distal portions of the device, it can be advantageous to exhibit high bending flexibility and low columnar stiffness to minimize potential injury to vascular tissue and/or to provide a readily shapeable distal tip to aid in navigation capabilities of the device.

The increased bending flexibility provided by a one-beam cut pattern can minimize or prevent the tube from deforming the shape of the coincident internal structures of the guidewire. For example, a shapeable core (e.g. stainless steel) disposed within the tube may be bent or curved (i.e., plastically deformed) to provide the tip of the guidewire with a desired shape. This is often done to enable the operator to “steer” the guidewire by pointing the shaped tip in the desired direction at a vascular branch before further advancing the device. In many instances, forces associated with elastic recovery of the tube, which is often formed from a superelastic material, will be imparted against the core and will tend to straighten out the shaped tip of the core. Using a one-beam section at the coincident portions of the tube beneficially reduces the recovery force imparted against the shaped core and allows the shaped core to better maintain its shape.

While it may be advantageous to include a one-beam section in a distal portion of the tube, more proximal sections of the tube can include multi-beam cut patterns, such as a two-beam cut pattern, to retain more torque transmission and columnar stiffness at proximal sections where the need for column buckling and shapeability are less pressing, yet where adequate column stiffness and torque transmission are beneficial.

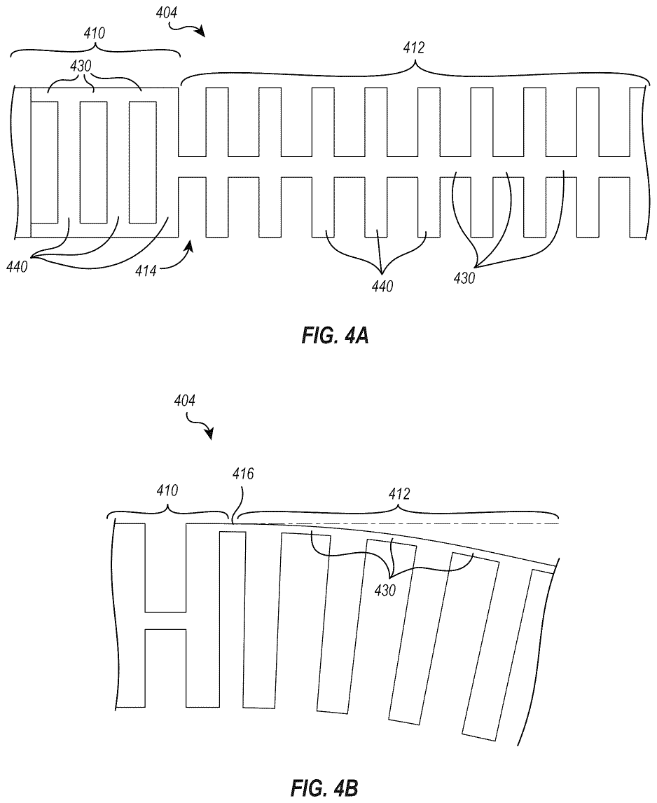

FIG. 4A illustrates a section of a tube 404 with a proximal section 410 of the tube 404 comprising a two-beam cut pattern and a distal section 412 comprising a one-beam cut pattern, with a transition point 414 between the proximal section 410 and distal section 412. The beams 430 are shown without a rotational offset for ease of illustration, though it will be understood that any of the rotational offset features disclosed herein may be utilized.

The mechanical properties of the tube 404 may change across the transition point 414 due to the different cut patterns disposed in proximal section 410 distal section 412. For example, bending flexibility may increase while torque transmission and columnar stiffness may decrease as the tube transitions from the two-beam cut pattern to the one-beam cut pattern. Such a transition may harm the performance of the device. For example, such a transition may concentrate mechanical stresses and/or may create an abrupt change in mechanical properties that interferes with the operator's tactile control of the device.

In some instances, such as when a plurality of one-beam cuts are aligned on one side of the tube, as illustrated in FIG. 4B, the beams 430 form a spine across the distal section 412. Such embodiments can create a preferred bend in the distal section, as is desired in some applications. However, such a configuration can cause or exacerbate a kink point 416 between the proximal section 410 and distal section 412 of the tube 404. The kink point 416 may concentrate stresses and/or decrease tactile control over the distal end of the device.

### Transition Section With Interspersed Cut Types

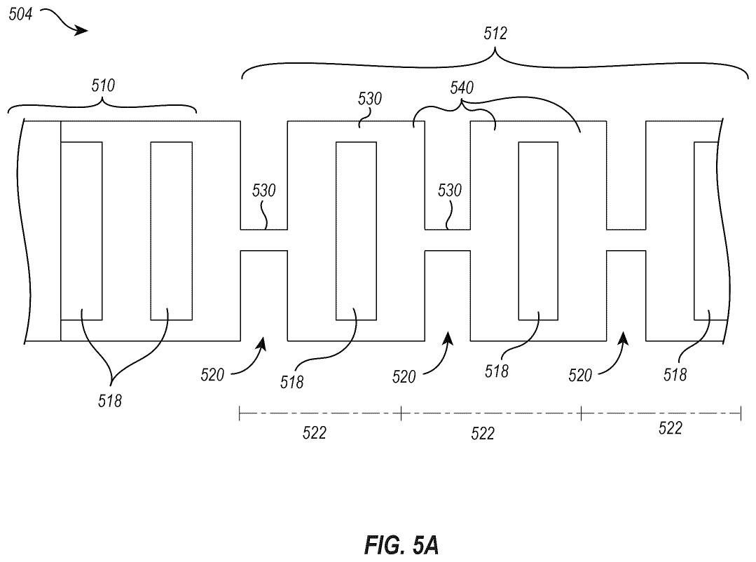

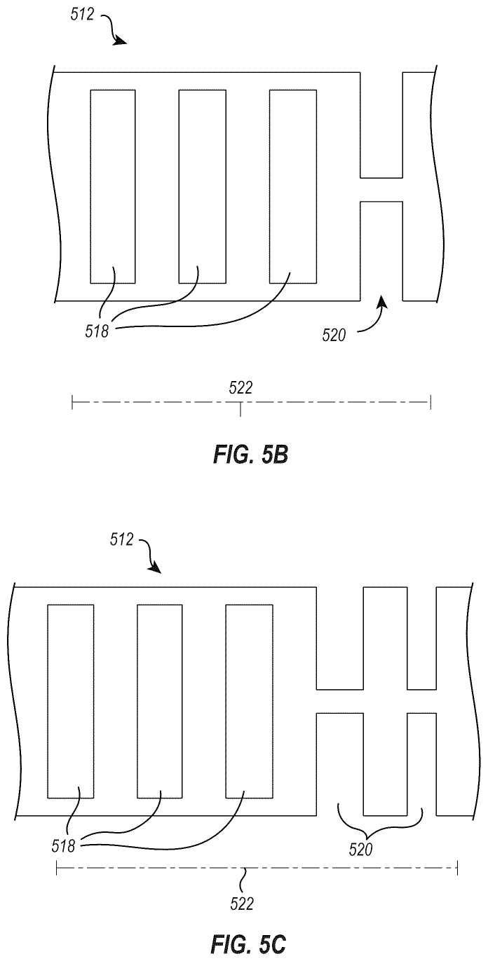

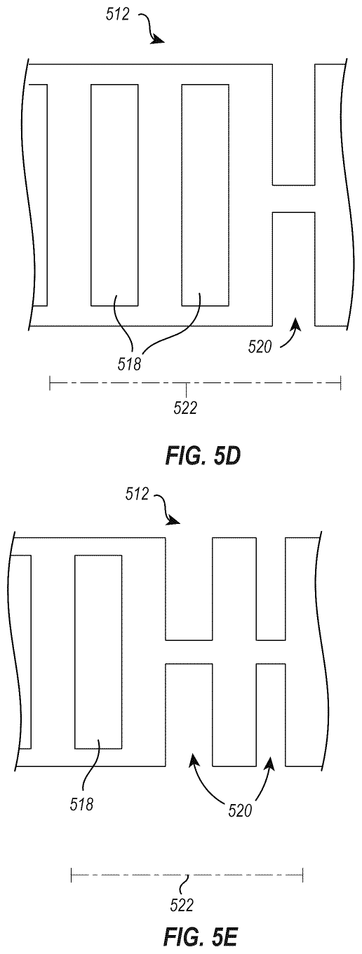

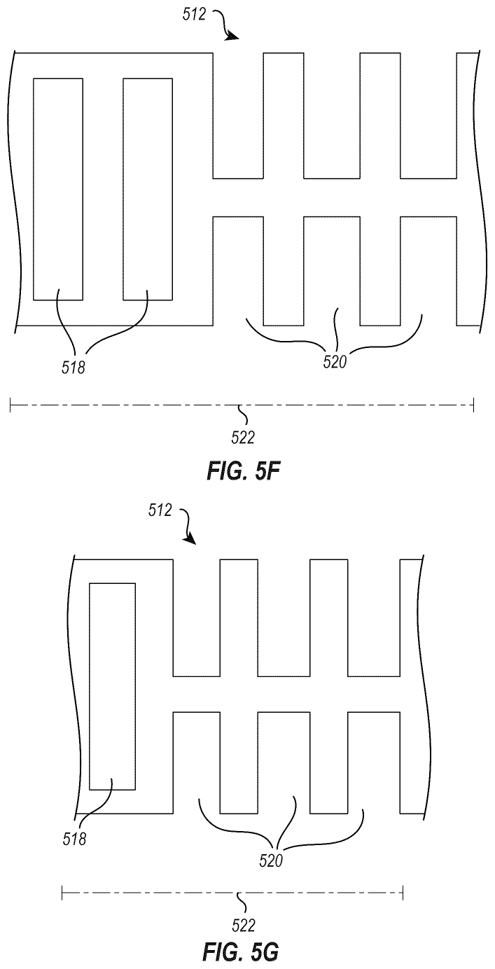

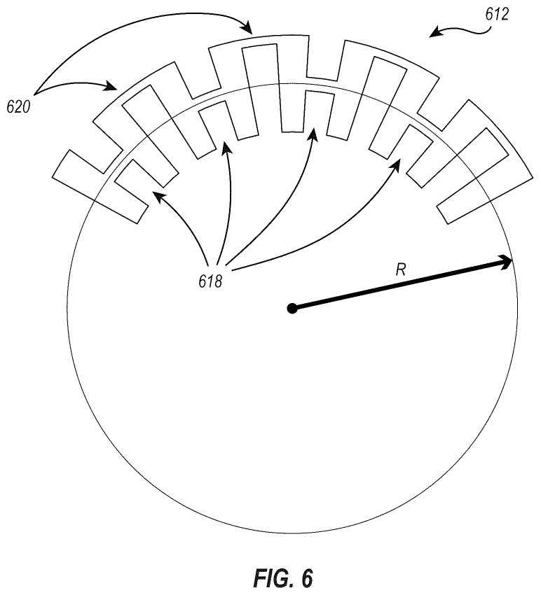

FIG. 5A illustrates a preferred embodiment that can provide a smooth a transition in bending flexibility, torque transmission, and/or columnar stiffness between proximal section 510 and distal section (not shown) of tube 504. Proximal section 510 comprises a series of two-beam cuts 518, the distal section comprises a series of one-beam cuts 520, and transition section 512 comprises interspersed two-beam cuts 518 and one-beam cuts 520. By interspersing both two-beam cuts 518 and one-beam cuts 520 in the transition section 512, the transition section 512 can blend properties of the two-beam cuts 518 with properties of the one-beam cuts 520 to gradually transition from a two-beam cut pattern to a one-beam cut pattern. This can beneficially minimize abrupt changes to bending flexibility, torque transmissibility, and columnar stiffness. Interspersion of different types of cuts in the transition section 512 beneficially spreads the local discontinuity over the length of the transition section 512.

Additionally, the benefits of the interspersed transition section 512 can be realized using cuts of substantially uniform spacing and beams of substantially uniform size. Prior approaches, for example, have attempted to smooth the transition from a two-beam cut pattern to a one-beam cut pattern by adjusting ring size (by varying longitudinal cut spacing) and/or beam size (by adjusting cut depth). However, while such approaches can smooth out changes in bending stiffness, they do not typically also smooth out changes in columnar stiffness and torque transmissibility.

Moreover, adjusting cut depth and/or spacing can introduce complexity to the manufacturing process, whereas forming a transition section with interspersed cut types can utilize the same cut parameters as used in other sections. Maintaining similar cut parameters (spacing, depth, etc.) can be beneficial in some applications. In some embodiments, however, a transition section can include both (i) adjusting ring size (by varying longitudinal cut spacing), (ii) adjusting beam size (by adjusting cut depth), and/or (iii) including interspersed cut types. Such embodiments can beneficially incorporate multiple separate approaches for smoothing the transition from one beam pattern type to the next. For example, a transition section can include, in a proximal to distal direction, a two-beam portion of relatively greater ring and/or beam size that transitions to a two-beam portion of relatively smaller ring and/or beam size that transitions to an interspersed pattern (e.g., 1:1 ratio and/or other ratios) that transitions to a one-beam portion of relatively greater ring and/or beam size that transitions to a one-beam portion of relatively smaller ring and/or beam size.

The interspersed two-beam and one-beam cuts may be organized in cut pattern comprising a plurality of units 522, where each unit 522 includes one or more instances of each cut type. FIG. 5A illustrates a transition section 512 where each unit 522 comprises two-beam cuts 518 and one-beam cuts 520 interspersed in a 1:1 ratio. In other words, transition section 512, as shown in FIG. 5A, comprises a plurality of repeating units 522 that consist of one one-beam cut 520 followed by one two-beam cut 518.

Units 522 may comprise different ratios of cut types. In one example, as illustrated in FIG. 5B, a unit 522 may include two-beam cuts 518 and one-beam cuts 520 that are interspersed in a 3:1 ratio, with three two-beam cuts 518 followed by a single one-beam cut 520.

Additionally, or alternatively, as illustrated in FIG. 5C, the transition section 512 can include a unit 522 comprising two-beam cuts 518 and one-beam cuts 520 that are interspersed in a 3:2 ratio, with three two-beam cuts 518 followed by two one-beam cuts 520.

Additionally, or alternatively, as illustrated in FIG. 5D, the transition section 512 can include a unit 522 comprising two-beam cuts 518 and one-beam cuts 520 that are interspersed in a 2:1 ratio, with two two-beam cuts 518 followed by a single one-beam cut 520.

Additionally, or alternatively, as illustrated in FIG. 5E, the transition section 512 can include a unit 522 comprising two-beam cuts 518 and one-beam cuts 520 that are interspersed in a 1:2 ratio, with a single two-beam cut 518 followed by two one-beam cuts 520.

Additionally, or alternatively, as illustrated in FIG. 5F, the transition section 512 can include a unit 522 comprising two-beam cuts 518 and one-beam cuts 520 that are interspersed in a 2:3 ratio, with two two-beam cuts 518 followed by three one-beam cuts 520.

Additionally, or alternatively, as illustrated in FIG. 5G, the transition section 512 can include a unit 522 comprising two-beam cuts 518 and one-beam cuts 520 that are interspersed in a 1:3 ratio, with a single two-beam cut 518 followed by three one-beam cuts 520.

Other proportions of two-beam cuts 518 and one-beam cuts 520 are possible. The order in which the two-beam cuts 518 and one-beam cuts 520 are arranged within a given unit 522 can also be varied. In presently preferred embodiments, a unit 522 typically includes 1-3 two-beam cuts 518 and 1-3 one-beam cuts 520. That is, the maximum number of longitudinal cut locations per unit 522 may be up to six, such as in a 3:3 ratio, with three two-beam cuts 518 followed by three one-beam cuts 520, for example. However, the maximum number of longitudinal cut locations per unit 522 may have other values and accordingly may be further increased, in some embodiments.

Units with different ratios of two-beam and one-beam cuts can be used to tailor the properties of a corresponding section of tube 504. For example, a section of tube 504 with a plurality of units 522 with two-beam cuts 518 and one-beam cuts 520 interspersed in a 3:1 ratio will result in a section of tube 504 that behaves more like a section comprising only two-beam cuts 518 than a section comprising only one-beam cuts 520. Accordingly, such a section of tube 504 will have less bending flexibility, increased torque transmissibility, and increased columnar stiffness when compared to an otherwise similar section that includes units with a greater proportion of one-beam cuts (e.g., with units comprising a 3:2, 2:1, 1:1, 1:2, 2:3, and/or 1:3 ratio of two-beam cuts to one-beam cuts). Accordingly, units with different ratios of cut types can be selectively positioned along a section of tube 504 to create a desired torquability, bending flexibility, and/or columnar stiffness gradient across a section of tube.

For example, in one embodiment, tube 504 comprises a proximal section 510 that includes a cut pattern of two-beam cuts 518, a distal section comprising a cut pattern of one-beam cuts 520, and a transition section 512 comprising a cut pattern that includes units 522 with 1-3 two-beam cuts 518 and 1-3 one-beam cuts 520. The units 522 closest to proximal section 510 may include two-beam cuts 518 and one-beam cuts 520 in a 3:1 ratio. Moving in the distal direction, later units 522 can include two-beam cuts 518 and one-beam cuts 520 in a 2:1 ratio, followed by units 522 that include two-beam cuts 518 and one-beam cuts 520 in a 1:1 ratio, followed by that include two-beam cuts 518 and one-beam cuts 520 in a 1:2 ratio followed by units 522 that include two-beam cuts 518 and one-beam cuts 520 in a 1:3 ratio. Such an embodiment creates a smooth gradient of bending flexibility, columnar stiffness, and torque transmissibility from the two-beam cut pattern of the proximal section 510 to the one-beam cut pattern of the distal section. Alternative embodiments may omit one or more of the foregoing unit configurations while still preserving an overall gradual transition.

In embodiments where several one-beam cuts are aligned along the same circumferential side of the tube to form a preferred bending direction, an interspersed configuration can be used to beneficially control the bending radius. FIG. 6 illustrates an embodiment where a transition section 612 includes two-beam cuts 618 (shown with one beam of each beam pair facing the view while the other beam is behind and hidden from view) and one-beam cuts 620 (in a 1:1 ratio). The one-beam cuts 620 are aligned on a single circumferential side of the transition section 612 to create a preferred bending direction. The bending radius “R” is primarily dependent on the bending that occurs at the one-beam cuts 620. As shown, the adjacent rings between a one-beam cut 620 can flex toward one another on the side opposite the beam. Adjusting the ratio of two-beam cuts 618 to one-beam cuts 620 provides a higher or lower proportion of one-beam cuts 620 to thereby adjust the affinity for the preferred bending radius.

Forming an interspersed configuration such as shown can beneficially impart a shape-set bend in the tube. That is, forming an interspersed configuration can introduce a shape-set bend in the tube such that the default shape of the tube (in the absence of counteracting forces) tends toward the bent shape. This can be desirable in certain applications.

### Additional Terms & Definitions

The different types of cuts described above may be arranged along a tube such that cuts form a cut pattern in a corresponding section of the tube. The term “cut pattern” is used herein to refer to a plurality of cuts along a tube. A cut pattern may comprise one or more types of cuts. For example, a cut pattern may comprise homogenous cuts (i.e., a plurality of cuts that are the same or substantially the same). Alternatively, a cut pattern may comprise more than one cut type interspersed along an axial direction of the tube. For example, the cut pattern may include one-beam cuts and two-beam cuts interspersed along an axial direction of the tube.

A “cut pattern” refers to a plurality of cuts of a given type along a given section of the tube. Thus, a plurality of successive one-beam cuts forms a “one-beam cut pattern”, a plurality of successive two-beam cuts forms a “two-beam cut pattern”, and so on. A section of the tube that includes a given cut pattern can be referred to according to that cut pattern. For example, a section of the tube that includes a one-beam cut pattern (and excludes other beam number cuts) can be referred to as a “one-beam section”, a section of the tube that includes a two-beam cut pattern (and excludes other beam number cuts) can be referred to as a “two-beam section”, and so on.

The term “cut” does not imply a specific method of manufacture to form the corresponding shape. Accordingly, the negative space of the “cut” may be formed by any suitable manufacturing method, including subtractive manufacturing methods such as micro-cutting or laser-cutting, or via an additive manufacturing method that directly forms the ring and beam structure (e.g., 3D printing methods), or combination thereof, for example.

The disclosure refers to the ability to avoid an “abrupt change” in bending flexibility, torque transmissibility (i.e., torquability), and/or columnar stiffness. While jumps in one or more of these parameters (i.e., change in stiffness) from one measured unit to the next may be discrete, the overall pattern of such jumps preferably approximates a linear series or a smooth curve. Thus, in the context of this disclosure, an “abrupt change” occurs where a jump from one unit to the next is greater than either immediately adjacent jump by a factor of more than about 1.5. An abrupt change is therefore avoided and the measure of the parameter is therefore “smooth” when no jump across units of the transition section is greater than either adjacent jump by a factor of more than about 1.5. Preferably, no jump across units of the transition section is greater than either adjacent jump by a factor of more than about 1.4, or 1.3, or 1.2.

The terms “approximately,” “about,” and “substantially” as used herein represent an amount or condition close to the stated amount or condition that still performs a desired function or achieves a desired result. For example, the terms “approximately,” “about,” and “substantially” may refer to an amount or condition that deviates by less than 10%, or by less than 5%, or by less than 1%, or by less than 0.1%, or by less than 0.01% from a stated amount or condition.

Elements described in relation to any embodiment depicted and/or described herein can be combined with elements described in relation to any other embodiment depicted and/or described herein. For example, the interspersed cut type features described in relation to FIGS. 5A-6 can be combined with any of the general device features describe in relation to FIGS. 1A-1B and/or the cut type features described in relation to FIGS. 2A-3D.

The present invention may be embodied in other forms, without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

1. An intravascular device comprising:
a tube comprising a proximal section, a distal section, and a transition section therebetween,
wherein the tube includes a plurality of cuts extending transversely into the tube, the plurality of cuts forming a plurality of circumferentially extending rings, each pair of adjacent rings being connected by one or more beams,
wherein the proximal section comprises a two-beam cut pattern in which two beams extend between and connect each pair of adjacent rings,
wherein the distal section comprises a one-beam cut pattern in which a single beam extends between and connects each pair of adjacent rings, and
wherein the transition section comprises a first type of cut that forms one beam between adjacent rings and a second type of cut that forms two beams between adjacent rings, wherein the first type of cut and second type of cut are interspersed with one another.

2. The device of claim 1, further comprising a core, wherein a distal section of the core passes into and is encompassed by the tube.

3. The device of claim 2, further comprising one or more coils disposed between an outer surface of the core and an inner surface of the tube.

4. The device of claim 1, wherein the transition section of the tube comprises a plurality of successive units, each unit comprising one to five cuts of the first type and one to five cuts of the second type.

5. The device of claim 4, wherein one or more units closer to the proximal section include more cuts of the second type than cuts of the first type as compared to one or more units closer to the distal section.

6. The device of claim 4, wherein one or more units closer to the distal section include more cuts of the first type than of the second type as compared to one or more units closer to the proximal section.

7. The device of claim 1, wherein the beams between rings formed by cuts of the first type are aligned along a single side of the tube.

8. The device of claim 1, wherein the beams between rings formed by cuts of the first type alternate in position from a first side of the tube to a second side of the tube.

9. The device of claim 1, wherein the tube is formed from a superelastic material.

10. The device of claim 1, wherein the plurality of cuts of the distal section of the tube are arranged with substantially uniform longitudinal spacing.

11. An intravascular device comprising:
a tube comprising a proximal section, a distal section, and a transition section therebetween,
wherein the tube includes a plurality of cuts extending transversely into the tube, the plurality of cuts forming a plurality of circumferentially extending rings, each pair of adjacent rings being connected by one or more beams,
wherein the proximal section comprises a cut pattern in which (X) beams extend between and connect each pair of adjacent rings,
wherein the distal section comprises a cut pattern in which (X-1) beams extend between and connect each pair of adjacent rings, and
wherein the transition section comprises a first type of cut that forms (X-1) beams between adjacent rings and a second type of cut that forms (X) beams between adjacent rings, wherein the first type of cut and second type of cut are interspersed with one another.

12. The device of claim 11, further comprising a core, wherein a distal section of the core passes into and is encompassed by the tube.

13. The device of claim 12, further comprising one or more coils disposed between an outer surface of the core and an inner surface of the tube.

14. The device of claim 11, wherein the transition section of the tube comprises a plurality of successive units, each unit comprising one to five cuts of the first type and one to five cuts of the second type.

15. The device of claim 14, wherein one or more units closer to the proximal section include more cuts of the second type than cuts of the first type as compared to one or more units closer to the distal section.

16. The device of claim 14, wherein one or more units closer to the distal section include more cuts of the first type than of the second type as compared to one or more units closer to the proximal section.

17. The device of claim 11, wherein the beams between rings formed by cuts of the first type are aligned along a single side of the tube.

18. The device of claim 11, wherein the beams between rings formed by cuts of the first type alternate in position from a first side of the tube to a second side of the tube.

19. The device of claim 11, wherein the tube is formed from a superelastic material.

20. The device of claim 11, wherein the plurality of cuts of the distal section of the tube are arranged with substantially uniform longitudinal spacing.
